(19) United States
(12) Patent Application Publication
(10) Pub. No.: US 2013/0210739 A1
(43) Pub. Date: Aug. 15, 2013

(54) BHLH PROTEINS AND THEIR USE AS DRUGS

(71) Applicants: Jean-Philippe Hugnot, Montpellier (FR); Pierre-Olivier Guichet, Montpellier (FR); Che Serguera, Paris (FR); Jacques Mallet, Paris (FR)

(72) Inventors: Jean-Philippe Hugnot, Montpellier (FR); Pierre-Olivier Guichet, Montpellier (FR); Che Serguera, Paris (FR); Jacques Mallet, Paris (FR)

(73) Assignees: UNIVERSITE PIERRE ET MARIE CURIE (PARIS 6), Paris (FR); UNIVERSITE MONTPELLIER 2 SCIENCES ET TECHNIQUES, Montpellier Cedex 5 (FR)

(21) Appl. No.: 13/811,440

(22) PCT Filed: Jul. 21, 2011
(86) PCT No.: PCT/EP2011/062581

§ 371 Date: Apr. 10, 2013

(30) Foreign Application Priority Data
Jul. 21, 2010 (EP) 10305804.6

(51) Int. Cl. A61K 38/17; A61K 45/06; A61K 31/7088

(52) U.S. Cl. CPC A61K 38/17; A61K 31/7088; A61K 45/06
USPC 514/17.7; 514/44 R

(57) ABSTRACT

The present invention relates to at least one protein belonging to the bHLH family and/or at least a nucleic acid molecule coding for the bHLH proteins as drug.

The present invention relates to bHLH proteins and their use as drugs.

Cancer stem cells (CSCs) are cancer cells (found within solid tumors or haematological cancers) that possess characteristics associated with normal stem cells, specifically the ability to give rise to all cell types found in determined cancer sample.

CSCs may generate tumors through the stem cell processes of self-renewal and differentiation into multiple cell types.

CSCs have been identified initially in leukaemia sample wherein an isolated subpopulation of leukaemic cells that express a specific surface marker CD34, but lacks the CD38 marker are capable of initiating tumors in NOD/SCID mice that is histologically similar to the donor.

The existence of leukaemic stem cells prompted further research into other types of cancer. CSCs have recently been identified in several solid tumors, including cancers of the breast, brain, colon, ovary, pancreas and prostate.

The efficacy of cancer treatments is measured by the reduction of the tumor mass. However, since CSCs form a small proportion of the tumor, they may not necessarily be targeted by the treatment. CSCs cells are proposed to persist in tumors as a distinct population and cause relapse and metastasis by giving rise to new tumors.

Therefore, there is a need to specifically eradicate cancer stem cells in order to limit relapse of tumors after remission, following antitumor therapies.

Gliomas are primitive tumours of the CNS which are derived from tumorigenesis of cells of the glial lineage (astrocytes and oligodendrocytes) (Louis, 2006, Annu Rev Pathol 1, 97-117; Behin, 2003, Lancet 361, 323-331). They are the most frequent brain tumours with an incidence of 1/20 000/inhabitants/year (3000 new cases in France, 15 000 news cases in US per year) (Bondy, 2008, Cancer. 2008 Oct. 1; 113(7 Suppl):1953-68; Bauchet 2007, J Neurooncol. 2007 September; 84(2):189-99). These tumors are aggressive, highly invasive and neurologically destructive.

Glioma are divided in two main categories:
High grade gliomas (grade III-IV according to WHO classification, Louis, 2007, Acta Neuropathol. 2007 August; 114(2):97-109), which are mostly represented by multiform glioblastomas (GBM). These tumours contain highly proliferating cells and are associated with a very poor prognosis.
Low grade gliomas (WHO grade II glioma, G2G), which growth slowly but which ineluctably evolves to anaplasia within 5-10 years.

One important feature of gliomas is their diffuse aspect due to migration and infiltration of the parenchyma from which deterioration will occur. There is currently no curative treatment for these tumors and despite maximum treatment efforts, median survival of patients diagnosed with GBM ranges from 9 to 12 months, a statistic that has changed very little in decades.

GBM are the most common glioma in humans (Kleihues 2000, Cancer. 2000 Jun. 15; Maher, 2001, Genes Dev. 2001 Jun. 1; 15(11):1311-33) and can evolve from low grade glioma (secondary GBM) or develop de novo (primary GBM). Like all cancers, GBM share a relatively restricted set of characteristics crucial to their phenotype: proliferation in the absence of external growth stimuli, avoidance of apoptosis and no limits to replication, escape from both external growth-suppressive forces and the immune response, formation of new blood vessels and the ability to invade normal tissues (Hanahan and Weinberg 2000, Cell. 100(1):57-70). Furthermore, despite their striking heterogeneity, common alterations in specific cellular signal transduction pathways occur within most GBMs (Louis, 2006, Annu Rev Pathol 1, 97-117).

GBM may be derived from transformation of differentiated cells or alternatively of adult stem/progenitor cells (Dai 2003, Cancer J 9, 72-81; Holland, 2001, Curr Opin Neurol 14, 683-688).

Indeed, GBMs contain 1-20% of cancer stem cells which grow on non adherent substrates to generate clonal expansion called neurospheres. The latter are multipotential and generate astrocytes and neuronal-like cells upon differentiation on adhesive substrate. These cancer stem cells appear to be more tumorigenic than the rest of tumoral cells when grafted in immunocompromised animals. In addition, these cells seem to be more chemo- and radio-resistant than the other tumoral cells.

As a consequence, new glioma drugs or treatments have to be found to specifically eradicate these cells.

Tumoral stem cells, like the non tumoral stem cells, reside in special vascular niches (Gilbertson, 2007, Nat Rev Cancer 7, 733-736) which provide high level of canonical stem cell signallings such as Wnt, Notch or SHH (Ischenko, 2008, Curr Med Chem. 2008; 15(30):3171-84). These pathways maintain the cells in an undifferentiated state and contribute to their self-renewal.

In addition, it is now well documented that GBM cancer stem cells rely on a special set of genes (for instance Sox2, Olig2, Bmi1 . . . ) to maintain a high level of self-renewal. These genes could be considered as potential targets to specifically eliminate these cells.

The number of new molecules specifically developed to cure gliomas is very low. Treatments of the proliferative tumoral cells transiently reduce tumor progression. However, a relapse occurs, due to the persistence of tumoral stem cells.

One possible approach is to differentiate these CSCs into post-mitotic cells so as to turn off the proliferation program, and therefore limiting tumor growth. This approach has very satisfying results in Acute Promyelocytic leukemia (APL) treated with retinoic acid and/or arsenic.

This strategy requires a broad knowledge of the mechanisms and/or genes which are responsible for the differentiation of normal and tumoral stem cells. This has been partially elucidated for stem cells of the nervous system. Indeed, during development, these stem cells differentiate into neuronal cells as a consequence of the stabilisation and the increase of neurogenic transcription factors, notably those belonging to the bHLH family and in particular NGN1, NGN2, NGN3 and ASCL1 (Bertrand, 2002, Nat Rev Neurosci. July; 3(7):517-30). This is well illustrated by gain and loss of function studies. Indeed, overexpression of NGN2 in the neural tube of chick embryos results in premature cell cycle exit and premature neuronal differentiation of neuroepithelial stem cells, (Mizugushi, 2001 Neuron 31:757-771; Sugimori 2007 Development, 134(8):1617-29). Conversely double mutant animals for NGN1 and 2 present a marked reduction in neurogenesis (Scardigli, 2001, Neuron 31203-217). Likewise, overexpression of ASCL1 in cerebellum promotes cell-cycle exit and differentiation (Alvarez-Rodriguez 2009 J Cell Sci. March 1; 122, 595-9) while ASCL1 loss of function results in impaired neurogenesis of postnatal neural stem cells (EMBO J. 2004 Nov. 10; 23(22):4495-505).

In this context, some of the above genes have been used for the treatment of different pathologies.

For instance, WO 2004/016779 discloses pharmaceutical compositions comprising NGN2 or ASCL1 for their use in cell therapy of neurological diseases.

US 2004/152168 proposes compositions comprising NGN2 or ASCL1 for treating insulin associated disorders.

WO 01/26643 discloses a method for inhibiting tumorigenic properties of melanoma cells by using bHLH proteins.

An alternative possibility to eliminate cancer stem cells is to inactivate one or several stem cell signallings with specific drugs targeting these pathways (Ischenko, 2008, Curr Med Chem. 2008; 15(30):3171-84).

Last, one can also consider the possibility of targeting key stem cell genes to eradicate the source of the tumor.

The need of an efficient drug able to completely and efficiently eradicate cancer cells, and cancer stem cells, remains.

Therefore, one aim of the invention is to provide a new efficient drug for treating pathologies, including cancer.

Another aim of the invention is to provide an efficient therapy for treating tumors, without risk of relapse.

The present disclosure relates to a composition comprising
at least one protein belonging to the bHLH family, chosen among NGN2, or ASCL1, and NEUROD1 and/or
at least a nucleic acid molecule coding for said bHLH proteins, i.e. coding for NGN2, or ASCL1, and NEUROD1 proteins, as drug inducing cell death, preferably as apoptotic drug.

The present invention relates to a composition comprising
at least one protein belonging to the bHLH family, chosen among NGN2, ASCL1, and NEUROD1 and/or
at least a nucleic acid molecule coding for said bHLH proteins, i.e. coding for NGN2, ASCL1, and NEUROD1 proteins, for its use for inducing apoptosis of cancer cells, preferably for inducing apoptosis of cancer stem cells.

In other word the invention relates to a composition comprising
either at least one protein belonging to the bHLH family, chosen among NGN2, ASCL1, and NEUROD1,
or at least a nucleic acid molecule coding for said bHLH proteins, i.e. coding for NGN2, ASCL1, and NEUROD1 proteins proteins,
or at least one protein belonging to the bHLH family, chosen among NGN2, ASCL1, and NEUROD1, and at least a nucleic acid molecule coding for said bHLH proteins, i.e. coding for NGN2, ASCL1, and NEUROD1 proteins,

for its use for inducing apoptosis of cancer cells, preferably for inducing apoptosis of cancer stem cells.

Terms “at least one” or “at least a” refer, in the invention, to one, or two, or three, or more.

The 63 possibilities, covered by the invention, are represented by the following table 1:

TABLE 1

| Composition | NGN2 | | | ASCL1 | | | NeuroD1 | | |
|---|---|---|---|---|---|---|---|---|---|
| n° | P | D | P + D | P | D | P + D | P | D | P + D |
| 1 | + | | | | | | | | |
| 2 | | + | | | | | | | |
| 3 | | | + | | | | | | |
| 4 | | | | + | | | | | |
| 5 | | | | | + | | | | |
| 6 | | | | | | + | | | |
| 7 | | | | | | | + | | |
| 8 | | | | | | | | + | |
| 9 | | | | | | | | | + |
| 10 | + | | | + | | | | | |
| 11 | + | | | | + | | | | |
| 12 | + | | | | | + | | | |
| 13 | + | | | | | | + | | |
| 14 | + | | | | | | | + | |
| 15 | + | | | | | | | | + |
| 16 | | + | | + | | | | | |
| 17 | | + | | | + | | | | |
| 18 | | + | | | | + | | | |
| 19 | | + | | | | | + | | |
| 20 | | + | | | | | | + | |
| 21 | | + | | | | | | | + |
| 22 | | | + | + | | | | | |
| 23 | | | + | | + | | | | |
| 24 | | | + | | | + | | | |
| 25 | | | + | | | | + | | |
| 26 | | | + | | | | | + | |
| 27 | | | + | | | | | | + |
| 28 | | | | + | | | + | | |
| 29 | | | | + | | | | + | |
| 30 | | | | + | | | | | + |
| 31 | | | | | + | | + | | |
| 32 | | | | | + | | | + | |
| 33 | | | | | + | | | | + |
| 34 | | | | | | + | + | | |
| 35 | | | | | | + | | + | |
| 36 | | | | | | + | | | + |
| 37 | + | | | + | | | + | | |
| 38 | + | | | | + | | + | | |
| 39 | + | | | | | + | + | | |
| 40 | + | | | + | | | | + | |
| 41 | + | | | | + | | | + | |
| 42 | + | | | | | + | | + | |
| 43 | + | | | + | | | | | + |
| 44 | + | | | | + | | | | + |
| 45 | + | | | | | + | | | + |
| 46 | | + | | + | | | + | | |
| 47 | | + | | | + | | + | | |
| 48 | | + | | | | + | + | | |
| 49 | | + | | + | | | | + | |
| 50 | | + | | | + | | | + | |
| 51 | | + | | | | + | | + | |
| 52 | | + | | + | | | | | + |
| 53 | | + | | | + | | | | + |
| 54 | | + | | | | + | | | + |
| 55 | | | + | + | | | + | | |
| 56 | | | + | | + | | + | | |
| 57 | | | + | | | + | + | | |
| 58 | | | + | + | | | | + | |
| 59 | | | + | | + | | | + | |
| 60 | | | + | | | + | | + | |
| 61 | | | + | + | | | | | + |
| 62 | | | + | | + | | | | + |
| 63 | | | + | | | + | | | + |

Table 1 represents the 63 possibilities of the composition used according to the invention. P: Protein; D: DNA; P+D: Protein+DNA.

The invention is based on the unexpected observation made by the Inventors that the over-expression of NGN2, ASCL1 or NEUROD1 proteins induces cell death. Surprisingly, other bHLH proteins such as NGN1 and NGN3 are not able to induce cell death when their expression is enforced in cells.

Moreover, the above mentioned over-expression of NGN2, ASCL1 or NEUROD1 proteins also induces enforced differentiation of tumoral cells into functional differentiated cells.

For instance, a tumor derived from central nervous system can be enforced to differentiate toward neurone that is electro-physiologically active (See example section).

In the invention, the expression “a nucleic acid molecule coding for bHLH proteins” refers to the NGN2, ASCL1 or NEUROD1 genes or the corresponding transcripts (RNA transcript of the NGN2, ASCL1, or NEUROD1 genes).

By convention, in the invention, the name of proteins is capitalized (e.g. NGN2) and the corresponding gene coding for said protein is represented by slanting characters (e.g. NGN2).

The composition used according to the invention kills cells, in particular tumoral cells, more preferably cancer stem cells.

There are many possibilities to kill cells: by inducing programmed cellular death (also called apoptosis), by inducing necrosis, or by inducing autophagy.

Autophagy, or autophagocytosis, is a catabolic process involving the degradation of a cellular own components through the lysosomal machinery. It is a tightly-regulated process that plays a normal part in cell growth, development, and homeostasis, helping to maintain a balance between the synthesis, degradation, and subsequent recycling of cellular products. It is a major mechanism by which a starving cell reallocates nutrients from unnecessary processes to more-essential processes.

Apoptosis is the process of programmed cell death that may occur in multicellular organisms. Biochemical events lead to characteristic cell changes (morphology) and death. These changes include blebbing, loss of cell membrane asymmetry and attachment, cell shrinkage, nuclear fragmentation, chromatin condensation, and chromosomal DNA fragmentation.

Necrosisis corresponds to the premature death of cells and living tissue. Necrosis is caused by factors external to the cell or tissue, such as infection, toxins, or trauma. This is in contrast to apoptosis, which is a naturally occurring cause of cellular death. While apoptosis often provides beneficial effects to the organism, necrosis is almost always detrimental and can be fatal.

The composition used according to the invention is able to kill cells rapidly and efficiently, as illustrated in the Example section.

The composition as defined above is used as apoptotic drug.

“Apoptotic drug” according to the invention defines a drug having properties to induce programmed cell death of cells treated with said drug.

Apoptosis can be easily measured by general protocols known in the art. These protocols include, for instance:
annexin V detection at the cell surface, by using flow cytometry
caspase activation measurement, such as Caspase 3 activation
DNA fragmentation measurement, for instance by TUNNEL method,
Cytochrome C measurement.

It is also possible to measure apoptosis by flow cytometry (FACS) by measuring the DNA content, in particular by quantifying the population of cells having a DNA content lower than the DNA content of a diploid cell (sub G1 population).

The skilled person is able also to measure cell apoptosis by other well described methods disclosed in the art.

In one advantageous embodiment, the invention relates to a composition used as defined above, wherein said NGN2 protein comprises or consists of
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1, or
a fragment of said amino acid molecules provided that said fragments induce cell death, in particular apoptosis, preferably said fragments comprising or being constituted by the amino acid sequence SEQ ID NO: 5.

In one other advantageous embodiment, the invention relates to the composition used as defined above, wherein said ASCL1 protein comprises or consists of
the amino acid sequence SEQ ID NO:2, or
any amino acid sequence having at least 85% of identity with the amino acid sequence SEQ ID NO:2, preferably any amino acid sequence having at least 90% of identity with the amino acid sequence SEQ ID NO:2,
a fragment of said amino acid molecules provided that said fragments induce cell death, in particular apoptosis, preferably said fragments comprising or being constituted by the amino acid sequence SEQ ID NO: 6.

In one advantageous embodiment, the invention relates to a composition used as defined above, wherein said NEUROD1 protein comprises or consists of
the amino acid sequence SEQ ID NO:9, or
any amino acid sequence having at least 85% of identity with the amino acid sequence SEQ ID NO:9, preferably any amino acid sequence having at least 90% of identity with the amino acid sequence SEQ ID NO:9, or
a fragment of said amino acid molecules provided that said fragments induce cell death, in particular apoptosis, preferably said fragments comprising or being constituted by the amino acid sequence SEQ ID NO: 11.

In another advantageous embodiment, the invention relates to a composition used as previously defined, wherein said nucleic acid molecule coding for NGN2 protein comprises or consists of
the nucleic acid sequence SEQ ID NO: 3, or
any nucleic acid molecule having at least 75%, preferably at least 85%, more preferably at least 95% of homology with the nucleic acid sequence SEQ ID NO: 3, or
a fragment of nucleic acid molecules provided that said fragments induce cell death, in particular apoptosis, preferably said fragments comprising or being constituted by the amino acid sequence SEQ ID NO 7, coding for the fragment comprising or being constituted by the amino acid sequence SEQ ID NO: 5 as defined above.

In another advantageous embodiment, the invention relates to a composition used as defined above, wherein said nucleic acid molecule coding for ASCL1 protein comprises or consists of
the nucleic acid sequence SEQ ID NO: 4, or
any nucleic acid molecule having at least 75%, preferably at least 85%, more preferably at least 95% of homology with the nucleic acid sequence SEQ ID NO: 4,
a fragment of said nucleic acid molecules provided that said fragments induce cell death, in particular apoptosis, preferably said fragments comprising or being constituted by the nucleic acid sequence SEQ ID NO 8, coding for the fragment comprising the amino acid sequence SEQ ID NO: 6 as defined above.

In another advantageous embodiment, the invention relates to a composition used as previously defined, wherein said nucleic acid molecule coding for NEUROD1 protein comprises or consists of
the nucleic acid sequence SEQ ID NO: 10, or
any nucleic acid molecule having at least 75%, preferably at least 85%, more preferably at least 95% of homology with the nucleic acid sequence SEQ ID NO: 10, or
a fragment of nucleic acid molecules provided that said fragments induce cell death, in particular apoptosis, preferably said fragments comprising or being constituted by the amino acid sequence SEQ ID NO 10, coding for the fragment comprising or being constituted by the amino acid sequence SEQ ID NO: 12 as defined above.

Another embodiment according to the invention relates to a composition used as defined above, wherein each of said nucleic acid molecule is comprised, or contained, in at least one vector, said vector comprising nucleic acid sequences allowing the expression of said nucleic acid molecule.

As mentioned above, the nucleic acid molecule coding for NGN2, ASCL1, or NEUROD1 proteins, or fragments thereof, as defined above, is contained in a vector containing specific sequences allowing:
its replication in a lower eukaryote, such as bacteria,
the initiation of transcription,
termination of transcription, and
antibiotics resistance, or drug susceptibility.

Moreover said vector contains any other sequences necessary for an expression in an host organism.

By “nucleic acid sequences allowing the expression of said nucleic acid molecule”, the invention refers to nucleic acid sequences allowing the initiation of transcription, i.e. promoter, and nucleic acid sequences allowing the termination of transcription.

In order to allow the expression in mammal cells, such as human cells, the nucleic acid sequence according to the invention can be placed under the control of promoter sequences such as Cytomegalovirus (CMV) promoter, Rous Sarcoma Virus (RSV) promoter, Elongation Factor 1α (EF1-α) promoter or Ubiquitin gene (Ub) promoter. All these promoters allow the expression whatsoever the cell.

The nucleic acid molecule mentioned above can be also placed under the control of Long Terminal repeat (LTR) promoters derived from retroviruses or lentiviruses.

All these promoters are known in the art, and the skilled person can easily choose the best promoter for controlling the expression of the nucleic acid molecules coding for NGN2, ASCL1, and NEUROD1 proteins or fragments thereof.

In the invention, vectors to deliver and express the nucleic acid molecule into tumoral stem cells are for instances, HIV-1 derived lentiviral vectors, either carrying a wild type or a mutant (D64V) integrase, leading respectively to integrating (Trip-PGK) and non-integrating (Ni-Trip-PGK) vectors (Sarky, 2008, Curr Gene Ther. 8(6):430-7).

Non integrative lentivirus allows gene transfer to the nucleus and its expression without integration in the genome. The viral genomes remain as episomes which lack replication signals and are thus progressively diluted out. This allows a “hit and run” mode of gene expression and represents a significant safety benefits over their integrative counterparts.

In addition to lentiviruses, adenovirus vectors such as canine adenovirus vectors (Paul, 2008, Cancer Biol Ther. 2008 May; 7(5):786-93), can be also used in the invention for targeting glioma cells.

In one other embodiment, the invention relates to a composition used as mentioned above, comprising a vector comprising:
a first nucleic acid molecule coding for said NGN2 protein, or fragments thereof, and
a second nucleic acid molecule coding for said ASCL1 protein, or fragments thereof,
wherein both first and second nucleic acid molecule are placed under the control of nucleic acid sequences allowing the expression of said nucleic acid molecules.

In this embodiment, both nucleic acid molecules are contained in the same vector and their expression is governed by one promoter. For this purpose, the above first and above second nucleic acid molecules are linked by an Internal Ribosomal Entry Site (IRES) sequence that allow the transcription in a unique transcript of 2 sequences, but will provide the transcription of two separated and independent proteins.

In one other embodiment, the invention relates to a composition used as mentioned above, comprising a vector comprising:
a first nucleic acid molecule coding for said NGN2 protein, or fragments thereof, and
a second nucleic acid molecule coding for said NEUROD1 protein, or fragments thereof,
wherein both first and second nucleic acid molecule are placed under the control of nucleic acid sequences allowing the expression of said nucleic acid molecules.

In this embodiment, both nucleic acid molecules are contained in the same vector and their expression is governed by one promoter. For this purpose, the above first and above second nucleic acid molecules are linked by an Internal Ribosomal Entry Site (IRES) sequence that allow the transcription in a unique transcript of 2 sequences, but will provide the translation of two separated and independent proteins.

In another embodiment, the invention relates to a composition used as mentioned above, comprising a vector comprising:
a first nucleic acid molecule coding for said ASCL1 protein, or fragments thereof, and
a second nucleic acid molecule coding for said NEUROD1 protein, or fragments thereof,
wherein both first and second nucleic acid molecule are placed under the control of nucleic acid sequences allowing the expression of said nucleic acid molecules.

In this embodiment, both nucleic acid molecules are contained in the same vector and their expression is governed by one promoter. For this purpose, the above first and above second nucleic acid molecules are linked by an Internal Ribosomal Entry Site (IRES) sequence that allow the transcription in a unique transcript of 2 sequences, but will provide the translation of two separated and independent proteins.

In still another embodiment, the invention relates to a composition used as mentioned above, wherein:
a first nucleic acid molecule coding for said NGN2 protein, or fragments thereof,
a second nucleic acid molecule coding for said ASCL1 protein, or fragments thereof, and
a third nucleic acid molecule coding for said NEUROD1 protein, or fragments thereof,
are comprised in the same vector,
wherein first, second and third nucleic acid molecule are placed under the control of nucleic acid sequences allowing the expression of said nucleic acid molecules.

In this embodiment, the three nucleic acid molecules are contained in the same vector and their expression is governed by one promoter. For this purpose, the above first, second and third second nucleic acid molecules are linked by an Internal Ribosomal Entry Site (IRES) sequence that allow the transcription in a unique transcript of 3 sequences, but will provide the translation of three separated and independent proteins.

In one other embodiment, the invention relates to a composition used as defined above, wherein
at least one protein belonging to the bHLH family, chosen among NGN2, ASCL1 and NEUROD1 and/or
at least a nucleic acid molecule coding for said bHLH proteins, are used in a simultaneous, separate or sequential manner.

In one other embodiment, the invention relates to a composition used corresponding to one of the compositions disclosed above in table 1, wherein the component of said composition are used in a simultaneous, separate or sequential manner.

Thus, in this embodiment, the invention relates to a composition used as defined above, comprising the following components:
NGN2 protein and ASCL1 protein, or
nucleic acid molecule coding for NGN2 protein and ASCL1 protein, or
NGN2 protein, nucleic acid molecule coding for NGN2 protein and ASCL1 protein, or
NGN2 protein and nucleic acid molecule coding for ASCL1 protein, or
nucleic acid molecule coding for NGN2 protein and nucleic acid molecule coding for ASCL1 protein, or
NGN2 protein, nucleic acid molecule coding for NGN2 protein and nucleic acid molecule coding for ASCL1 protein, or
NGN2 protein, ASCL1 protein and nucleic acid molecule coding for ASCL1 protein, or
nucleic acid molecule coding for NGN2 protein, ASCL1 protein and nucleic acid molecule coding for ASCL1 protein, or
NGN2 protein, nucleic acid molecule coding for NGN2 protein, ASCL1 protein and nucleic acid molecule coding for ASCL1 protein,
said components being used in a simultaneous, separate or sequential manner.

In one other embodiment, the invention relates to the use as defined above of a composition corresponding to one of the compositions 10 to 63 disclosed above in table 1, wherein the component of said composition are used in a simultaneous, separate or sequential manner.

From table 1, the skilled person knows what are the components of said composition. For instance;
composition 10 of table 1 comprises: NGN2 and ASCL1 protein,
composition 24 of table 1 comprises: NGN2 protein and nucleic acid coding for NGN2 protein, and ASCL1 protein and nucleic acid coding for ASCL1 protein,
composition 47 of table 1 comprises: nucleic acid coding for NGN2 protein, nucleic acid coding for ASCL1 protein, and NEUROD1 protein,
composition 59 of table 1 comprises: NGN2 protein and nucleic acid coding for NGN2 protein, and nucleic acid coding for ASCL1 protein, and NEUROD1 protein and nucleic acid coding for NEUROD1 protein, and
composition 63 of table 1 comprises: NGN2 protein and nucleic acid coding for NGN2 protein, and ASCL1 protein and nucleic acid coding for ASCL1 protein, and NEUROD1 protein and nucleic acid coding for NEUROD1 protein.

In one other embodiment, the invention relates to a composition used as defined above, further comprising at least one antitumoral agent.

By “antitumoral agent”, it is defined in the invention a compound or a drug commonly used for treating cancer in the frame of chemotherapy.

The majority of antitumoral agents can be divided into alkylating agents, antimetabolites, anthracyclines, plant alkaloids, topoisomerase inhibitors. These agents commonly interfere with cell division and DNA replication, and therefore limiting the multiplication of cancer cells.

Some advantageous antitumoral agents used according to the invention are cisplatin, vincristin, vinblastin, taxanes compounds or ectoposides.

In still another embodiment, the invention relates to a composition used as defined above, wherein
at least one protein belonging to the bHLH family, chosen among NGN2, ASCL1 and NEUROD1, and/or
at least a nucleic acid molecule coding for said bHLH proteins, and
said least one antitumoral agent are used in a simultaneous, separate or sequential manner.

Advantageously, the invention relates to a composition used as defined above, wherein said composition comprises:

either
at least two protein belonging to the bHLH family, chosen among NGN2, ASCL1, and NEUROD1,
or
at least two nucleic acid molecule coding for said bHLH proteins, or
at least one protein belonging to the bHLH family, chosen among NGN2, ASCL1, and NEUROD1, and
at least a nucleic acid molecule coding for said bHLH proteins, which are used in a simultaneous, separate or sequential manner.

Terms “at least two protein belonging to the bHLH family, chosen among NGN2, ASCL1, and NEUROD1” means NGN2 and ASCL1 protein, NGN2 and NEUROD1 proteins, ASCL1 and NEUROD1 proteins and NGN2, ASCL1 and NEUROD1 proteins.

Terms “at least two nucleic acid molecule coding for said bHLH proteins” means nucleic acid molecules coding for NGN2 and ASCL1 protein, nucleic acid molecules coding for NGN2 and NEUROD1 proteins, nucleic acid molecules coding for ASCL1 and NEUROD1 proteins and nucleic acid molecules coding for NGN2, ASCL1 and NEUROD1 proteins.

Another embodiment according to the invention relates to a composition used as previously defined, in association with a pharmaceutically acceptable carrier.

The appropriate pharmaceutically acceptable carrier is determined by the skilled person.

For instance, if the composition used according to the invention contains proteins, said composition can be in a form of liposome, microsphere carriers, or the protein can be in a form of fusion protein with VIH TAT protein or Protein Transduction Domain (PTD) of viral proteins.

The above mentioned carriers are such that they allow the delivery to, and the entry into, the target cell of the protein contained in the composition used according to the invention.

In particular, pharmaceutically acceptable carrier allows crossing the blood brain barrier (BBB).

For instance, nucleic acid molecules are encapsulated in a 100 nm pegylated liposome and conjugated with receptor specific targeting monoclonal antibodies can cross the BBB and target tumoral cells (Pardridge, 2007, Pharm Res. 24(9):1733-44)

For instance, if the composition used according to the invention contains nucleic acid molecules, carriers can be liposome, microsphere carriers or inactivated viral particles, that allow the penetration of the nucleic acid molecule into the target cell.

These examples are not limitative.

Dosage of the active substance depends on the administration route, and can be easily determined by a skilled person. The pharmaceutical composition used according to the invention can be administered by intravenous route, sub-cutaneous route, systemic route, or can be administered locally by infiltration, or per os.

The pharmaceutical composition used according to the invention can be administered at a dosage from about 0.001 g/kg/day to about 0.1 g/kg/day, according to the administration route.

In particular, the pharmaceutical compositions according to the invention may be administered at a dosage from about 0.05 to about 5 g/day in adults, or from about 0.01 to about 1 g/day for children.

In a particular embodiment, the pharmaceutical composition used according to the invention contains at least a compound as previously defined in a form of the pharmaceutically acceptable salts known to a person skilled in the art, such as sodium salts, ammonium salts, calcium salts, magnesium salts, potassium salts, acetate salts, carbonate salts, citrate salts, chloride salts, sulphate salts, amino chlorhydate salts, borhydrate salts, phosphate salts, dihydrogenophosphate salts, succinate salts, citrate salts, tartrate salts, lactate salts, mandelate salts, methane sulfonate salts (mesylate) or p-toluene sulfonate salts (tosylate).

The invention also relates to a composition, preferably an apoptotic composition, as defined above, for its use for the treatment of central nervous system (CNS) tumors and neuroendocrine tumors.

Also, the invention relates to the use of a composition, preferably an apoptotic composition, as defined above, for the preparation of a drugs intended for the treatment of central nervous system (CNS) tumors and neuroendocrine tumors.

The invention also relates to a composition, preferably an apoptotic composition, as defined above, comprising
at least one protein belonging to the bHLH family, chosen among NGN2, ASCL1 and NEUROD1, or fragment thereof, and/or
at least a nucleic acid molecule coding for said bHLH proteins, i.e. coding for NGN2, ASCL1 and NEUROD1 proteins, or fragment thereof, for its use for the treatment of central nervous system (CNS) tumors and neuroendocrine tumors.

An advantageous embodiment of the invention relates to a composition as defined above, wherein

said NGN2 protein comprises or consists of
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1, or
a fragment of said amino acid molecules provided that said fragments induce cell death, in particular apoptosis, preferably said fragments comprising or being constituted by the amino acid sequence SEQ ID NO: 5,
and/or
said ASCL1 protein comprises or consists of
the amino acid sequence SEQ ID NO:2, or
any amino acid sequence having at least 85% of identity with the amino acid sequence SEQ ID NO:2, preferably any amino acid sequence having at least 90% of identity with the amino acid sequence SEQ ID NO:2,
a fragment of said amino acid molecules provided that said fragments induce cell death, in particular apoptosis, preferably said fragments comprising or being constituted by the amino acid sequence SEQ ID NO: 6,
and/or
said NEUROD1 protein comprises or consists of
the amino acid sequence SEQ ID NO:9, or
any amino acid sequence having at least 85% of identity with the amino acid sequence SEQ ID NO:9, preferably any amino acid sequence having at least 90% of identity with the amino acid sequence SEQ ID NO:9, or
a fragment of said amino acid molecules provided that said fragments induce cell death, in particular apoptosis, preferably said fragments comprising or being constituted by the amino acid sequence SEQ ID NO: 11,
and/or
said nucleic acid molecule coding for NGN2 protein comprise or consists of
the nucleic acid sequence SEQ ID NO: 3, or
any nucleic acid molecule having at least 75%, preferably at least 85%, more preferably at least 95% of homology with the nucleic acid sequence SEQ ID NO: 3,
a fragment of nucleic acid molecules provided that said fragments induce cell death, in particular apoptosis, preferably said fragments comprising or being constituted by the amino acid sequence SEQ ID NO 7, coding for the fragment comprising or being constituted by the amino acid sequence SEQ ID NO: 5 as defined above,
and/or
said nucleic acid molecule coding for ASCL1 protein comprise or consists of
the nucleic acid sequence SEQ ID NO: 4, or
any nucleic acid molecule having at least 75%, preferably at least 85%, more preferably at least 95% of homology with the nucleic acid sequence SEQ ID NO: 2,
a fragment of said nucleic acid molecules provided that said fragments induce cell death, in particular apoptosis, preferably said fragments comprising or being constituted by the nucleic acid sequence SEQ ID NO 8, coding for the fragment comprising the amino acid sequence SEQ ID NO: 6 as defined above,
and/or
said nucleic acid molecule coding for NEUROD1 protein comprise or consists of
the nucleic acid sequence SEQ ID NO: 10, or
any nucleic acid molecule having at least 75%, preferably at least 85%, more preferably at least 95% of homology with the nucleic acid sequence SEQ ID NO: 10,
a fragment of nucleic acid molecules provided that said fragments induce cell death, in particular apoptosis, preferably said fragments comprising or being constituted by the amino acid sequence SEQ ID NO 10, coding for the fragment comprising or being constituted by the amino acid sequence SEQ ID NO: 12 as defined above.

Another advantageous embodiment of the invention relates to the composition used as defined above, wherein said composition comprises

either each of said at least a nucleic acid molecule is comprised, or contained, in at least one vector, said vector comprising nucleic acid sequences allowing the expression of said nucleic acid molecule, or at least a vector comprising:
a first nucleic acid molecule coding for said NGN2 protein, or fragments thereof, and
a second nucleic acid molecule coding for said ASCL1 protein, or fragments thereof,
wherein both first and second nucleic acid molecule are placed under the control of nucleic acid sequences allowing the expression of said nucleic acid molecules, or
at least a vector comprising:
a first nucleic acid molecule coding for said NGN2 protein, or fragments thereof, and
a second nucleic acid molecule coding for said NEUROD1 protein, or fragments thereof,
wherein both first and second nucleic acid molecule are placed under the control of nucleic acid sequences allowing the expression of said nucleic acid molecules, or
at least a vector comprising:
a first nucleic acid molecule coding for said NEUROD1 protein, or fragments thereof, and
a second nucleic acid molecule coding for said ASCL1 protein, or fragments thereof,
wherein both first and second nucleic acid molecule are placed under the control of nucleic acid sequences allowing the expression of said nucleic acid molecules, or
at least a vector comprising:
a first nucleic acid molecule coding for said NGN2 protein, or fragments thereof,
a second nucleic acid molecule coding for said ASCL1 protein, or fragments thereof, and
a third nucleic acid molecule coding for said NEUROD1 protein, or fragments thereof,
wherein said first, second and third nucleic acid molecules are placed under the control of nucleic acid sequences allowing the expression of said nucleic acid molecules.

The invention relates to the use of the above composition for the preparation of a drug in the frame of the treatment of central nervous system (CNS) tumors and neuroendocrine tumors, said composition comprising:
at least one protein belonging to the bHLH family, chosen among NGN2, ASCL1, and NEUROD1 and/or
at least a nucleic acid molecule coding for said bHLH proteins, as defined above, advantageously in the advantageous embodiments.

The invention relates to a composition as defined above for its use for the treatment, or for the preparation of a drug in the frame of the treatment of central nervous system (CNS) tumors and neuroendocrine tumors.

Advantageous embodiment of the invention relates to
a composition used as defined above, or
the use of said composition for the preparation of a drug as defined above, or
wherein said CNS tumors are chosen among the group consisting of grade II-IV glioma according to the 2007 WHO classification of tumours of the central nervous system.

The WHO classification is known in the art (Acta Neuropathol. 2007 August; 114(2):97-109). Grade II-IV glioma refer to grade II glioma, grade Ill glioma and grade IV glioma.

Advantageous embodiment of the invention relates to
a composition used as defined above, or
the use of said composition for the preparation of a drug as defined above,
wherein said neuroendocrines tumors are chosen among the group consisting of primary or metastatic gastro-entero-pancreatic neuroendocrine tumors (Massironi, 2008, World J Gastroenterol. 14(35):5377-84) as defined by the WHO classification (Solcia, 2000, Berlin: Springer; pp. 56-70).

The invention relates to a method for treating central nervous system (CNS) tumors and neuroendocrine tumors, in particular grade II-IV glioma according to the 2007 WHO classification of tumours of the central nervous system or primary or metastatic gastro-entero-pancreatic neuroendocrine tumors, comprising the administration to a patients in a need thereof of a pharmaceutically effective amount of a composition comprising
at least one protein belonging to the bHLH family, chosen among NGN2, ASCL1 and NEUROD1, or fragment thereof, and/or
at least a nucleic acid molecule coding for said bHLH proteins, i.e. coding for NGN2, ASCL1 and NEUROD1 proteins, or fragment thereof.

An advantageous embodiment of the invention also relates to a method as defined above, wherein

said NGN2 protein comprises or consists of
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1, or
a fragment of said amino acid molecules provided that said fragments induce cell death, in particular apoptosis, preferably said fragments comprising or being constituted by the amino acid sequence SEQ ID NO: 5,
and/or
said ASCL1 protein comprises or consists of
the amino acid sequence SEQ ID NO:2, or
any amino acid sequence having at least 85% of identity with the amino acid sequence SEQ ID NO:2, preferably any amino acid sequence having at least 90% of identity with the amino acid sequence SEQ ID NO:2,
a fragment of said amino acid molecules provided that said fragments induce cell death, in particular apoptosis, preferably said fragments comprising or being constituted by the amino acid sequence SEQ ID NO: 6,
and/or
said NEUROD1 protein comprises or consists of
the amino acid sequence SEQ ID NO:9, or
any amino acid sequence having at least 85% of identity with the amino acid sequence SEQ ID NO:9, preferably any amino acid sequence having at least 90% of identity with the amino acid sequence SEQ ID NO:9, or
a fragment of said amino acid molecules provided that said fragments induce cell death, in particular apoptosis, preferably said fragments comprising or being constituted by the amino acid sequence SEQ ID NO: 11,
and/or
said nucleic acid molecule coding for NGN2 protein comprise or consists of
the nucleic acid sequence SEQ ID NO: 3, or
any nucleic acid molecule having at least 75%, preferably at least 85%, more preferably at least 95% of homology with the nucleic acid sequence SEQ ID NO: 3,
a fragment of nucleic acid molecules provided that said fragments induce cell death, in particular apoptosis, preferably said fragments comprising or being constituted by the amino acid sequence SEQ ID NO 7, coding for the fragment comprising or being constituted by the amino acid sequence SEQ ID NO: 5 as defined above,
and/or
said nucleic acid molecule coding for ASCL1 protein comprise or consists of
the nucleic acid sequence SEQ ID NO: 4, or
any nucleic acid molecule having at least 75%, preferably at least 85%, more preferably at least 95% of homology with the nucleic acid sequence SEQ ID NO: 2,
a fragment of said nucleic acid molecules provided that said fragments induce cell death, in particular apoptosis, preferably said fragments comprising or being constituted by the nucleic acid sequence SEQ ID NO 8, coding for the fragment comprising the amino acid sequence SEQ ID NO: 6 as defined above,
and/or
said nucleic acid molecule coding for NEUROD1 protein comprise or consists of
the nucleic acid sequence SEQ ID NO: 10, or
any nucleic acid molecule having at least 75%, preferably at least 85%, more preferably at least 95% of homology with the nucleic acid sequence SEQ ID NO: 10,
a fragment of nucleic acid molecules provided that said fragments induce cell death, in particular apoptosis, preferably said fragments comprising or being constituted by the amino acid sequence SEQ ID NO 10, coding for the fragment comprising or being constituted by the amino acid sequence SEQ ID NO: 12 as defined above.

The present invention will be illustrated by the following figures and the following examples.

### FIGURES

FIG. 1 represents the expression of NGN2 and SOX2 genes, measured by QPCR, in glioma tumors. The columns represent the means of NGN2 and SOX2 expression in 20 tumors of each grade (light grey columns=grade II glioma, dark grey columns=grade III glioma and black columns=grade IV glioma) and 20 non tumoral brains (white columns). Y axis represents arbitrary units on a logarithmic scale of QPCR quantification. This graph indicates that glioma expresses a low level of NGN2 and in contrast a high level of SOX2, a gene typically expressed by undifferentiated stem cells. Non parametric Kruskal and Wallis H tests were used to compare gene expression medians. Error bars represent standard error of mean (sem). Significances: *** (p<0.001).

FIG. 2 represents the expression of NGN2 and SOX2 genes, measured by QPCR, in 5 different GBM stem cell primary cultures namely, Gli1 (white columns), Gli2 (light grey columns), Gli3 (middle grey columns), Gli4F11 (dark grey columns) and clonal Gli4F11 (black columns). Y axis represents arbitrary units on a logarithmic scale of QPCR quantification. This graph indicates that GBM stem cells express a low level of NGN2 and in contrast a higher level of SOX2, a gene typically expressed by undifferentiated stem cells.

FIG. 3 represents the number of cells remaining one week after infection of GBM stem cells (Gli4F11) cultured as neurospheres, with increasing amount of integrative or non integrative NGN2 or GFP expressing lentivirus. This graph indicates that either integrative or non integrative NGN2 lentiviruses eliminate the vast majority of GBM stem cells. X-axis represents ng of p24 protein per 10⁴ cells and Y-axis the number of cell per well. Curve with black lozenges represents cells infected with integrative lentivirus expressing GFP, curve with black triangles represents cells infected with non integrative lentivirus expressing GFP, curve with black circles represents cells infected with integrative lentivirus expressing NGN2 and curve with black squares represents cells infected with non integrative lentivirus expressing NGN2.

FIGS. 4A and 4B represent photographs of GBM stem cells (Gli4F11) cultured as neurospheres infected with lentivirus, 7 days after infection. Scale bar=200 μm.

FIG. 4A represents neurospheres obtained from cells infected with control lentivirus (GFP)

FIG. 4B represents neurospheres obtained from cells infected with lentivirus allowing the expression of NGN2.

FIG. 5 corresponds to a graph representing the effect of NGN2 or ASCL1 lentivirus infection on GBM stem cell number. First column represents the number of cells when non infected. Column 2 represents the number of cells when infected with integrative lentivirus expressing GFP. Column 3 represents the number of cells when infected with integrative lentivirus expressing NGN2. Column 4 represents the number of cells when infected with integrative lentivirus expressing ASCL1. Column 5 represents the number of cells when infected with non integrative lentivirus expressing GFP. Column 6 represents the number of cells when infected with non integrative lentivirus expressing NGN2. Column 7 represents the number of cells when infected with a non integrative lentivirus expressing ASCL1. These results were obtained using 100 ng of p24 proteins per 10⁵ cells.

FIG. 6 represents the percentage of apoptotic cells (TUNEL positive cells) 24, 48 or 72 h after viral infection using 100 ng of p24 protein per 10⁵ cells. In each group, first column (black column) represents the number of TUNEL positive cells infected by integrative GFP, second column (dark grey column) represents the number of TUNEL positive cells infected by integrative NGN2, third column (light grey column) represents the number of TUNEL positive cells infected by non integrative GFP and fourth column (white column) represents the number of TUNEL positive cells infected by non integrative NGN2.

FIG. 7 corresponds to a graph representing the percentage of proliferating cells, as measured by KI67 immunofluorescence, after infection with integrative lentivirus (using 100 ng of p24 protein per 10⁵ cells) expressing GFP (column 1) or expressing NGN2 (column 2).

FIG. 8 illustrates the predominant action of NGN2 over that of NGN1 and NGN3. The graph represents the number of GBM stem cells remaining 20 days after the infection of integrative lentiviruses (using 100 ng of p24 protein per 10⁵ cells). Column 1 represents cells infected with GFP-expressing lentivirus; column 2 represents cells infected with NGN1-expressing lentivirus, column 3 represents cells infected with NGN2-expressing lentivirus and column 4 represents cells infected with NGN3-expressing lentivirus.

FIGS. 9 A and B represent photographs of remaining GBM stem cells (Gli4F11), grown on an adherent substrate (laminin), 10 days after infection.

FIG. 9 A represents cells infected with GFP-expressing lentivirus.

FIG. 9 B represents cells infected with NGN2-expressing lentivirus. NGN2 cells have a typical neuronal morphology.

FIG. 10 represents the detection, by immunofluorescence, of the expression of the typical neuronal protein Doublecortin (Dcx) in NGN2 infected cells (white cytoplasmic staining).

FIGS. 11 A and B illustrate the electrophysiological analysis of GFP and NGN2 infected cells.

FIG. 11 A shows a large inward current (arrow) detected in NGN2 infected cells, but not in control GFP infected cells.

FIG. 11 B represents action potential recordings from the same cells. Only NGN2 infected cells were able to trigger an action potential (arrow) (mean depolarizing current was 0.45±0.10 nA, n=4). Increasing the amplitude of depolarizing current to 1.5-2 nA failed to elicit electrical activity in GFP infected cells.

FIG. 12 represents the percentage of cells from Gli4, Gli5, Gli7 polyclonal lines remaining 7 days after lentiviral infection using 100 ng of p24 protein per 10⁵ cells. In each group, first column (dark grey column) represents the number of cells infected by integrative GFP, second column (light grey column) represents the cells infected by integrative NGN2. Y axis represents the percentage of cells. Stars indicate significant Mann-Whitney test (p<0.01).

### EXAMPLES

### Example 1

### Effect of NGN20R ASCL10R NEUROD1 Expression on Tumoral Cell Death

Material and Methods.

Cellular Model

The effect of surexpressing NGN2, ASCL1 and NEUROD1 cDNAs in GBM stem cells was investigated using four cancer stem cell lines (Gli4F11, Gli4, Gli5, Gli7) which was derived in the lab from patients diagnosed with a high grade glioma tumor (grade IV according to WHO classification).

These lines grow and self-renew in non adherent conditions and are able to form clonal neurospheres when seeded at one cell in 96 wells dish.

These lines have an abnormal caryotype.

These lines express typical cancer stem cell markers (nestin, CD133, CD15) and contain a side population (Hoescht exclusion).

These lines are multipotential and generate GFAP+, Map2ab+ and GalC+ cells after differentiation.

These line generate highly infiltrating high grade tumors in NOD/SCID mice. After 4 months, the whole brain is invaded by tumoral cells which remain proliferative.

Cell Culture

Gli4F11, Gli4, Gli5, Gli7 cells are cultured in serum-free DMEM/F12 media supplemented with non vitamin A-B27 (2%) and N2 (1%) serum-replacement media (Invitrogen), FGF2, EGF2 (10 ng/ml, Peproteck), Heparine (2 μg/ml, Sigma), Ciprofloxaxine (2 μg/ml, Euromedex), Gentamycine (10 μg/ml, Fisher), fungin (10 μg/ml, Cayla) and fungizone (0.25 μg/ml, Fisher). The cell are passaged classically using complete dissociation with trypsine/EDTA 0, 25% for 3′ followed by trypsin inactivation with trypsin inhibitor (Sigma, T9003). Cells are either grown free-floating on non-adherent substrate coated flasks (poly-2-hydroxyethylmethacrylate, poly HEMA from Sigma) or adherent substrate coated dishes (poly-D-lysine (25 μg/ml) and laminin (2 μg/cm2, Sigma)).

Lentivirus Construction

The human NGN2, ASCL1 and NEUROD1 cDNAs were cloned between the house keeping promoter of the phosphoglycerate kinase gene (PGK) and the Woodchuck hepatitis virus post-transcriptional regulating element (WPRE) in the plasmid Trip-PGK-WPRE coding for a self inactivating HIV-1 derived vector containing a central cPPT sequence (Zennou V, et al. (2001) Nat Biotechnol 19(5):446-450). The inventors produced two types of HIV-1 derived vectors, either carrying a wild type or a mutant (D64V) integrase, leading respectively to integrating (Trip-PGK-Ngn2) and non-integrating (Ni-Trip-PGK-ngn2) vector phenotypes (Sarkis C, et al. (2008) Curr Gene Ther 8(6):430-437). Stocks of vectors were prepared by transitory transfection of 293T cells with the p8.91 encapsidation plasmid (ΔVprΔVifΔVpuΔNef) (Zufferey R, et al. (1997) Nat Biotechnol 15(9):871-875) either with a wt or a mutated integrase, pHCMV-G encoding the VSV envelope and a plasmid containing the vector, as described previously (Zennou V, et al. (2001) Nat Biotechnol 19(5):446-450). Briefly, the supernatants were treated with DNAse I prior to ultracentrifugation then resuspended in PBS and frozen (−80°) until use. The amount of p24 capsid protein was determined by HIV-1 core profile ELISA (NEN) as described by the manufacturer.

Results

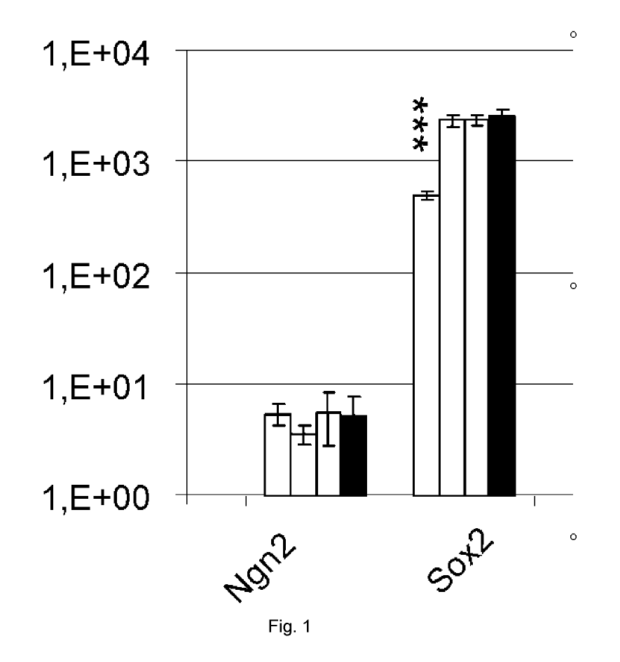

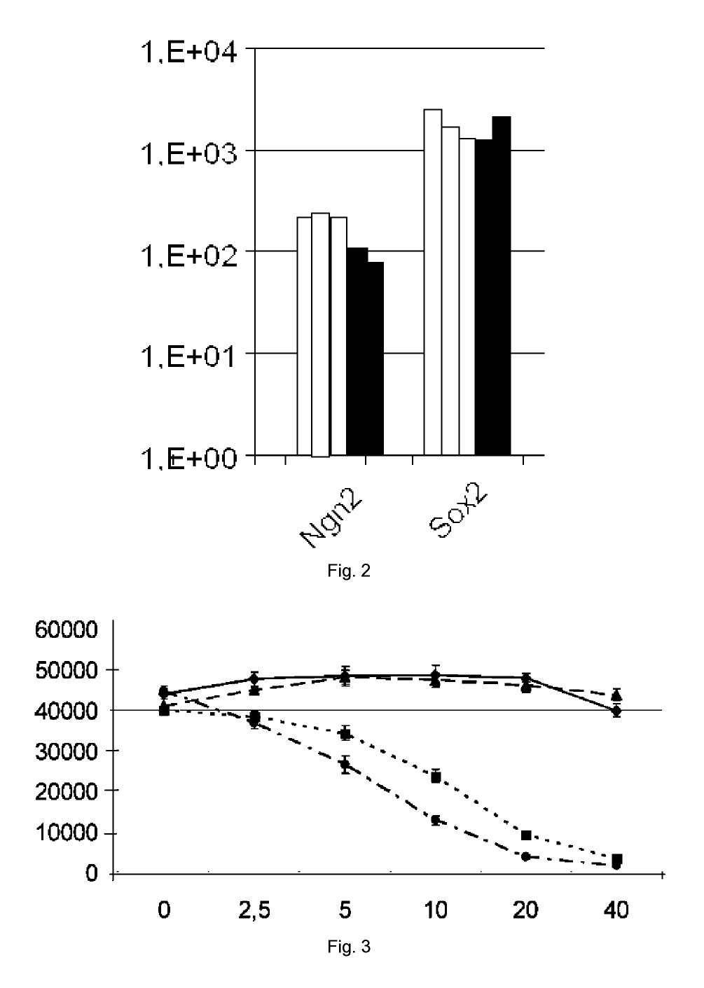

NGN2 Expression in Glioma (FIGS. 1 and 2)

FIGS. 1 and 2 illustrate that NGN2 is weakly expressed in glioma tumors (grade II to IV) and in five GBM stem cell cultures compared to SOX2, a gene important for maintaining GBM stem cell undifferentiated. This shows the predominance of an undifferentiatied state in glioma and GBM stem cells.

Effect of Lentiviral Infection on Cell Number (FIG. 3, 4, 5)

Non integrating or integrating lentiviruses expressing NGN2, ASCL1 or GFP (control) were used to infect Gli4F11 GBM cells growing as neurospheres (non adherent condition).

First, cells dissociated with trypsin were seeded at 10 000 cells per well in poly HEMA-coated 24 wells plate containing 1 ml of growth media and lentiviral particles (between 0 to 40 ng of p24 protein per well, 4 wells per conditions). After seven days, the cell number was determined by direct trypsinization of the cells by adding 300 μl of Trypsin 2.5% (sigma, 4799) into the well. After 10 minutes at 37° C., the cells were triturated and their number was counted using Z2 coulter counter (Beckman) using a 10-20 μm window.

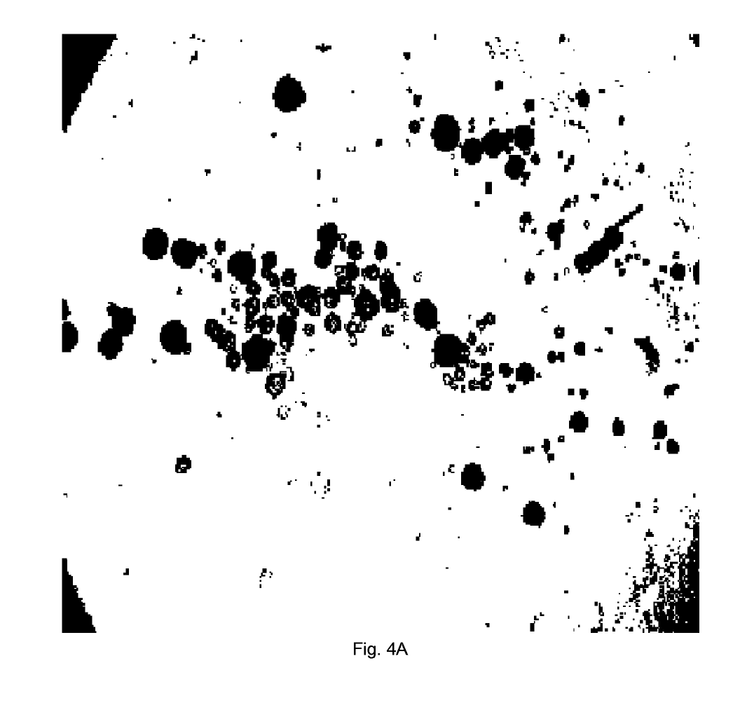

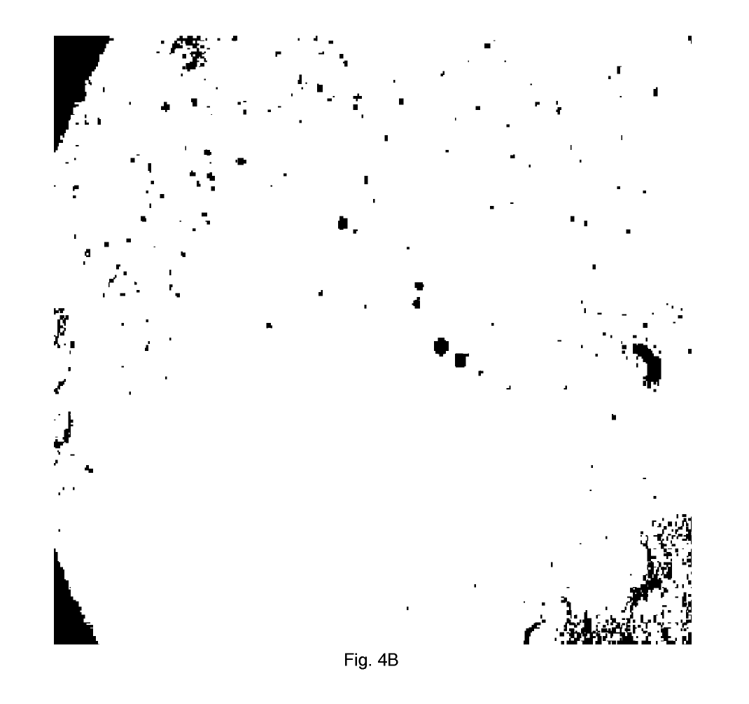

Results are shown in FIGS. 3, 4A and B and 5.

FIG. 3 shows that compared to control GFP lentiviruses, NGN2 integrative or non integrative lentiviruses induce a massive reduction of the GBM stem cells number.

FIGS. 4 A and B illustrates the aspect of cultures, 7 days after treatment with GFP or NGN2 lentiviruses. Note the almost complete disappearance of neurospheres on the right photograph (B).

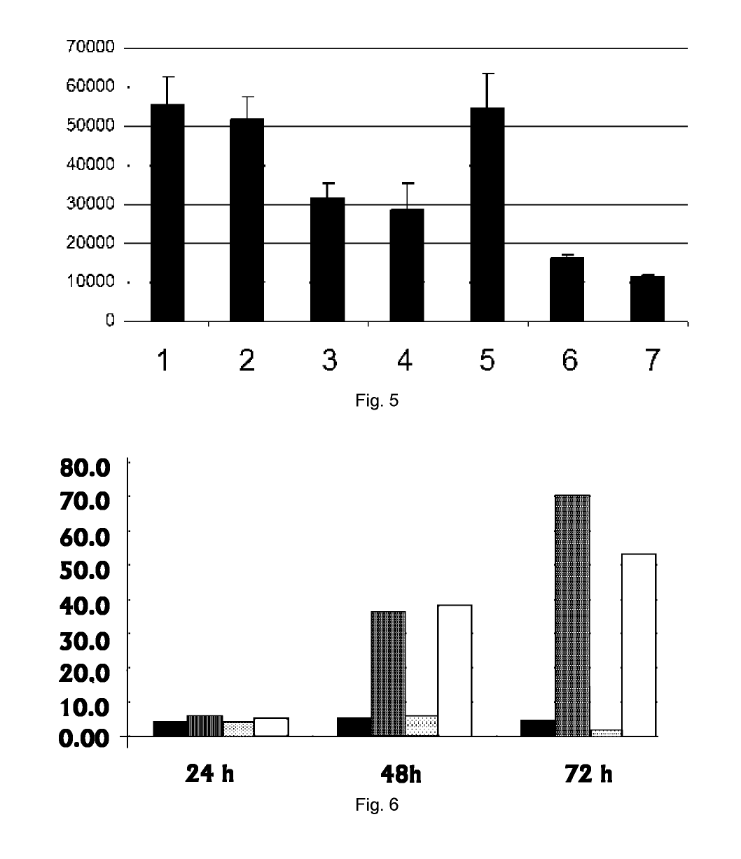

FIG. 5 shows that ASCL1 is as efficient as NGN2 as reducing the cell number. Compared to GFP-lentivirus treated cells (column 2 for integrative virus and 5 for non integrative virus), both NGN2 (column 3 for integrative virus and 6 for non integrative virus) and ASCL1 (column 4 for integrative virus and 7 for non integrative virus) dramatically reduced the cell number.

Determination of the Rate of Apoptosis Induced by the Lentiviral Infection (FIG. 6)

To assess the rate of apoptosis induced by the lentiviral infection, the cells were seeded at 150 000 cells per wells in a 24-wells plate (containing poly D-Lysine/Laminin coated coverslips) with 1 ml of media and lentiviral particles (150 ng of p24 protein). At 24 h, 48 and 72 h after the infection, the cells were fixed with an ethanol:acetic acid 2:1 (v:v) solution. Apoptotic cells were detected using the TUNEL method with an Apoptag red in situ apotosis detection kit (Millipore) according to the manufacturer's protocol. Results are shown on FIG. 6.

48 hours post infection, a massive apoptosis (up to 40% of cells) is detected in cells infected with integrative or non integrative NGN2 lentiviruses compared to cells infected with GFP lentiviruses. The rate of apoptosis is further increased at 72 h post infection to reach 50-60% of the cells.

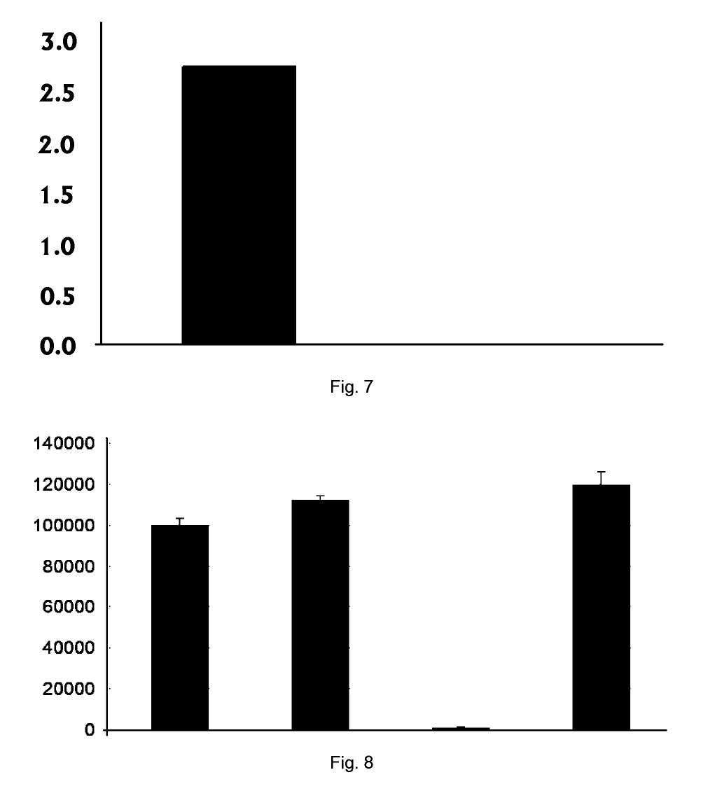

Determination of the Proliferation Inhibition Induced by the Lentiviral Infection (FIG. 7)

Cells were seeded with the same conditions as those used for apoptosis detection. After 7 days, the cells were fixed with 4% paraformaldehyde and the rate of proliferation was measured using detection of the nuclear KI67 (monoclonal antibody BD) antigen by classical immunofluorescence. Results are shown on FIG. 7. Compared to cells infected with a GFP integrative lentivirus, virtually no dividing KI67⁺ cells can be detected in NGN2 lentivirus treated cells.

Specific Effect of NGN2 over NGN1 and NGN3 Lentiviruses (FIG. 8)

GBM stem cells were infected with the same amount of p24 protein of NGN1, NGN2 and NGN3 integrative lentiviruses, then the cell number was determined after 20 days. FIG. 8 shows that NGN1 and NGN3 were not able to reduce the cell number compared to NGN2 lentiviruses. This illustrates the specific effect of the NGN2 sequence to induce GBM stem cell reduction.

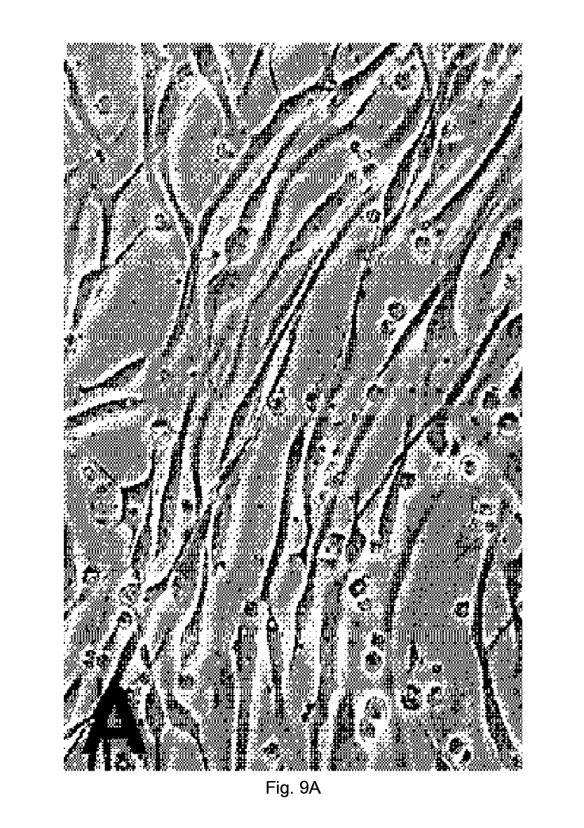

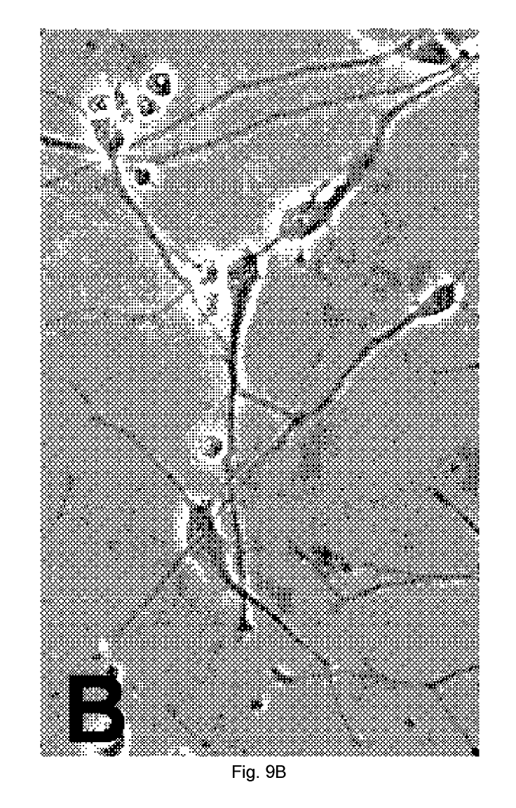

Forced Expression of NGN2 in GBM Stem Cells Induces Neuronal Differentiation (FIG. 9, 10, 11)

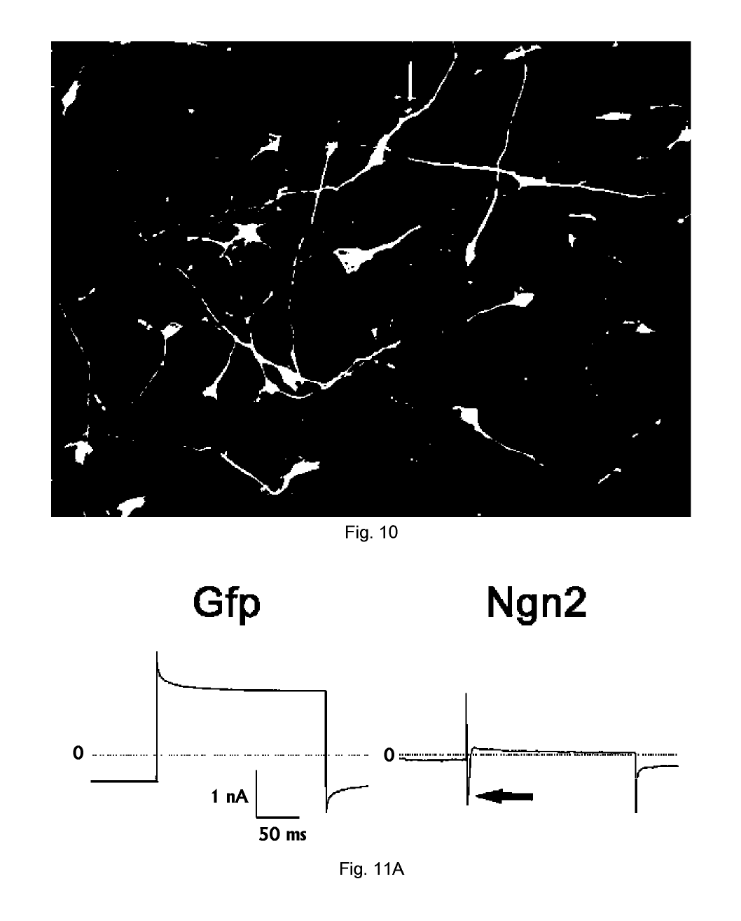

In addition to cell death, NGN2 lentivirus infection induces a striking cell morphology change. This is illustrated on FIG. 9 which shows that cells infected with GFP lentiviruses display a bipolar fusiform morphology (A) while those infected with NGN2 lentiviruses have a typical neuronal morphology (B). The neuronal phenotype of NGN2 infected cells is also demonstrated by the high expression of the neuronal specific marker Doublecortin (Dcx) detected by immunofluorescence (white cytoplasmic staining on FIG. 10). This neuronal phenotype is also supported by electrophysiology analysis which shows that a large inward current (arrow) was detected in 6 out of 7 recorded cells infected with NGN2 lentiviruses (mean peak amplitude was −515±125 pA) whereas control cells infected with GFP lentiviruses only elicited outward currents (FIG. 11A). In addition, FIG. 11B shows that only NGN2 lentivirus infected cells were able to trigger an action potential (arrow) (mean depolarizing current was 0.45±0.10 nA, n=4). Increasing the amplitude of depolarizing current to 1.5-2 nA failed to elicit electrical activity in GFP lentivirus infected cells. For NeuroD1, all the results obtained with this lentivirus are similar to those obtained with NGN2 and ASCL1 viruses

### Example 2

### Inhibition of Polyclonal Glioma Cell Growth by Ngn2 Overexpression

In addition to the clonal Gli4F11 cell line, the effect of overexpressing Ngn2 was extended in 3 others glioma cultures.

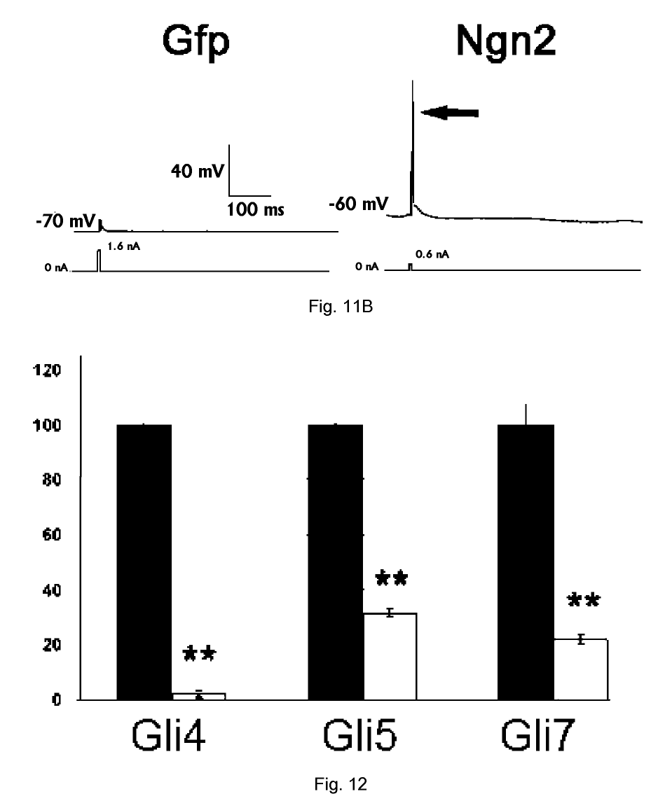

The Inventors confirmed their results by using the primary polyclonal Gli4 culture from which Gli4F11 was derived and by deriving two new cultures (Gli5 and Gli7) from two patients affected by glioblastoma. Like Gli4F11, these three lines present a phenotype reminiscent of proneural cells as evidenced by the expression of AscI1, NG2/CSPG4, Nkx2.2, Olig1/2 and PDGFRα markers. These cultures are multipotent, have abnormal karyotypes and form highly infiltrative Olig2⁺ Nkx2.2⁺ tumors similar to Gli4F11 when grafted in immunocompromised mice. In these 3 cultures, overexpression of Ngn2 using lentivirus, drastically reduced the cell number compared to control condition (GFP) after 5 days of culture, which was associated with an overt cell death, as shown in FIG. 12. Similar results were obtained in Gli5 and Gli7 cells by using ASCL1 and NEUROD1 lentivirus.

1-15. (canceled)

16. A method for treating central nervous system (CNS) tumors and neuroendocrine tumors, comprising the administration to a patients in a need thereof of a pharmaceutically effective amount of a composition comprising:
at least one protein belonging to the bHLH family, chosen among NGN2, ASCL1, and NEUROD1 and/or
at least a nucleic acid molecule coding for said bHLH protein.

17. The method according to claim 16, wherein said NGN2 protein comprises or consists of:
the amino acid sequence SEQ ID NO:1, or
any amino acid sequence having at least 85% of identity with the amino acid sequence SEQ ID NO:1, preferably any amino acid sequence having at least 90% of identity with the amino acid sequence SEQ ID NO:1.

18. The method according to claim 16, wherein said ASCL1 protein comprises or consists of:
the amino acid sequence SEQ ID NO:2, or
any amino acid sequence having at least 85% of identity with the amino acid sequence SEQ ID NO:2, preferably any amino acid sequence having at least 90% of identity with the amino acid sequence SEQ ID NO:2.

19. The method according to claim 16, wherein said NEUROD1 protein comprises or consists of:
the amino acid sequence SEQ ID NO:9, or
any amino acid sequence having at least 85% of identity with the amino acid sequence SEQ ID NO:9, preferably any amino acid sequence having at least 90% of identity with the amino acid sequence SEQ ID NO:9.

20. The method according to claim 16, wherein said nucleic acid molecule coding for NGN2 protein comprises or consists of:
the nucleic acid sequence SEQ ID NO: 3, or
any nucleic acid molecule having at least 75%, preferably at least 85%, more preferably at least 95% of homology with the nucleic acid sequence SEQ ID NO: 3.

21. The method according to claim 16, wherein said nucleic acid molecule coding for ASCL1 protein comprises or consists of:
the nucleic acid sequence SEQ ID NO: 4, or
any nucleic acid molecule having at least 75%, preferably at least 85%, more preferably at least 95% of homology with the nucleic acid sequence SEQ ID NO: 4.

22. The method according to claim 16, wherein said nucleic acid molecule coding for NEUROD1 protein comprises or consists of:
the nucleic acid sequence SEQ ID NO: 10, or
any nucleic acid molecule having at least 75%, preferably at least 85%, more preferably at least 95% of homology with the nucleic acid sequence SEQ ID NO: 10.

23. The method according to claim 16, wherein each of said at least nucleic acid molecule is comprised in one vector, said vector comprising nucleic acid sequences allowing the expression of said nucleic acid molecule.

24. The method according to claim 16, wherein
a first nucleic acid molecule coding for said NGN2 protein,
a second nucleic acid molecule coding for said ASCL1 protein, and
a third nucleic acid molecule coding for said NEUROD1 protein,
are comprised in the same vector,
said first, second and third nucleic acid molecules being are placed under the control of nucleic acid sequences allowing the expression of said nucleic acid molecules.

25. The method according to claim 16, wherein said composition comprises:
either at least two proteins belonging to the bHLH family, chosen among NGN2, ASCL1, and NEUROD1,
or at least two nucleic acid molecules coding for said bHLH proteins,
or at least one protein belonging to the bHLH family, chosen among NGN2, ASCL1, and NEUROD1, and at least a nucleic acid molecule coding for said bHLH proteins,
which are used in a simultaneous, separate or sequential manner.

26. The method according to claim 16, further comprising the administration of at least one antitumoral agent.

27. The method according to claim 16, wherein said CNS tumors are chosen among the group consisting of grade II-IV glioma according to the 2007 WHO classification of tumours of the central nervous system.

28. The method according to claim 16, wherein said neuroendocrine tumors are chosen among the group consisting of primary or metastatic Gastro-entero-pancreatic neuroendocrine tumors.


SEQUENCE LISTING (12 sequences)

SEQ ID NO: 1 - protein, 272 aa, Homo sapiens
MFVKSETLELKEEEDVLVLLGSASPALAALTPLSSSADEEEEEEPGASGGARRQRGAEAGQGARGGVAAGAEGCRPARLLGLVHDCKRRPSRARAVSRGAKTAETVQRIKKTRRLKANNRERNRMHNLNAALDALREVLPTFPEDAKLTKIETLRFAHNYIWALTETLRLADHCGGGGGGLPGALFSEAVLLSPGGASAALSSSGDSPSPASTWSCTNSPAPSSSVSSNSTSPYSCTLSPASPAGSDMDYWQPPPPDKHRYAPHLPIARDCI

SEQ ID NO: 2 - DNA, 2370 bp, Homo sapiens
cgcagccactgaaccacaagcagcttcgcgttaactggagtgcctgggagtcgcgtgccaggagccgcacggccagggactgactgacagacagacacgcaccaccaccacaacacacgagacccgggcgggccgccgccgccgccgccggggctcttggcaaactcgccggtcgcagaggtcccccgcggagctgcgccacagtagcgccgggcttgcagctttcacgccgggcgaaggacccggcgctgcgctcgcagctgcgcggagattcccggcacaggccaaagtcacagcaacgctgaggcacagttagagccaactaagatgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggcgccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctagagctgccatttctgctacccacgccaggccttagtgggttccctttcctgtccccagtcgagccctcctcccttcccctgcccctcctttccacgccctggaaaccatctcacttcacagggcaggtgtagcctttctgattcctcggttgtttcttgcatttcttggctttgggtatccttcattcagacgggctctgatttactgaaggtgtgatggagcttattgtcaaagccaagggtggcgttttgggggcgcttcttgagacgaaaaagaccctgggaagagatgatggtggcatatctaaagagtttgcagagcggactgacgctcctcccctttctctttaacgccgaaggacttggtgcagttcgtgtgaatctcacagggggaatgcaactggttcctgtgatctcttcacctttgcttctacatagagatgttaatgtcgagtagaaagaaatgtatcttagcatctgaatgattttgctggtaataatattatccacagatttgcaatggctggcatctgctttattcccattgctgtctgcaggctgtgggaatttcacctgtcaaaccaaacttccctctctgatgtgcactttgttctgtttcccagattcgtcacaatgcctattgtcctgtccttctctttcctttttcttccccattttgccatctgtctcttatgatttataaggggaaaaaaacttgttttgttagaggggcaggttagaagtcattgtataatttgtaggctttgtaatgattgaatgcaagcgtggaaatttaggctgaactctctatcaaaaggaaaaatgtggaggaaaagggaaaaatcaggagggaggattgcctcatgtattatttatttcgaccttttaggggagaaggaactcccccattctttcaagagattaaaaataaatcaacagtctgaaaacctaagcagacacggagcattatccggatcagccacacacgtgttcccttctatttattataaagaaatttttcatgggaaaatatgtattttttgtatattctacagagtttattctagtatgtatttacatcttgaagaacaagaaagttgttcttgtgattaaactataaataaactatctaattttcataaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - protein, 236 aa, Homo sapiens
MESSAKMESGGAGQQPQPQPQQPFLPPAACFFATAAAAAAAAAAAAAQSAQQQQQQQQQQQQAPQLRPAADGQPSGGGHKSAPKQVKRQRSSSPELMRCKRRLNFSGFGYSLPQQQPAAVARRNERERNRVKLVNLGFATLREHVPNGAANKKMSKVETLRSAVEYIRALQQLLDEHDAVSAAFQAGVLSPTISPNYSNDLNSMAGSPVSSYSSDEGSYDPLSPEEQELLDFTNWF

SEQ ID NO: 4 - DNA, 2490 bp, Homo sapiens
agcactctctcacttctggccagggaacgtggaaggcgcaccgacagggatccggccagggagggcgagtgaaagaaggaaatcagaaaggaagggagttaacaaaataataaaaacagcctgagccacggctggagagaccgagacccggcgcaagagagcgcagccttagtaggagaggaacgcgagacgcggcagagcgcgttcagcactgacttttgctgctgcttctgcttttttttttcttagaaacaagaaggcgccagcggcagcctcacacgcgagcgccacgcgaggctcccgaagccaacccgcgaagggaggaggggagggaggaggaggcggcgtgcagggaggagaaaaagcattttcactttttttgctcccactctaagaagtctcccggggattttgtatatattttttaacttccgtcagggctcccgcttcatatttccttttctttccctctctgttcctgcacccaagttctctctgtgtccccctcgcgggccccgcacctcgcgtcccggatcgctctgattccgcgactccttggccgccgctgcgcatggaaagctctgccaagatggagagcggcggcgccggccagcagccccagccgcagccccagcagcccttcctgccgcccgcagcctgtttctttgccacggccgcagccgcggcggccgcagccgccgcagcggcagcgcagagcgcgcagcagcagcagcagcagcagcagcagcagcagcaggcgccgcagctgagaccggcggccgacggccagccctcagggggcggtcacaagtcagcgcccaagcaagtcaagcgacagcgctcgtcttcgcccgaactgatgcgctgcaaacgccggctcaacttcagcggctttggctacagcctgccgcagcagcagccggccgccgtggcgcgccgcaacgagcgcgagcgcaaccgcgtcaagttggtcaacctgggctttgccacccttcgggagcacgtccccaacggcgcggccaacaagaagatgagtaaggtggagacactgcgctcggcggtcgagtacatccgcgcgctgcagcagctgctggacgagcatgacgcggtgagcgccgccttccaggcaggcgtcctgtcgcccaccatctcccccaactactccaacgacttgaactccatggccggctcgccggtctcatcctactcgtcggacgagggctcttacgacccgctcagccccgaggagcaggagcttctcgacttcaccaactggttctgaggggctcggcctggtcaggccctggtgcgaatggactttggaagcagggtgatcgcacaacctgcatctttagtgctttcttgtcagtggcgttgggagggggagaaaaggaaaagaaaaaaaaaagaagaagaagaagaaaagagaagaagaaaaaaacgaaaacagtcaaccaaccccatcgccaactaagcgaggcatgcctgagagacatggctttcagaaaacgggaagcgctcagaacagtatctttgcactccaatcattcacggagatatgaagagcaactgggacctgagtcaatgcgcaaaatgcagcttgtgtgcaaaagcagtgggctcctggcagaagggagcagcacacgcgttatagtaactcccatcacctctaacacgcacagctgaaagttcttgctcgggtcccttcacctcctcgccctttcttaaagtgcagttcttagccctctagaaacgagttggtgtctttcgtctcagtagcccccaccccaataagctgtagacattggtttacagtgaaactatgctattctcagccctttgaaactctgcttctcctccagggcccgattcccaaaccccatggcttccctcacactgtcttttctaccattttcattatagaatgcttccaatcttttgtgaattttttattataaaaaatctatttgtatctatcctaaccagttcggggatatattaagatatttttgtacataagagagaaagagagagaaaaatttatagaagttttgtacaaatggtttaaaatgtgtatatcttgatactttaacatgtaatgctattacctctgcatattttagatgtgtagttcaccttacaactgcaattttccctatgtggttttgtaaagaactctcctcataggtgagatcaagaggccaccagttgtacttcagcaccaatgtgtcttactttatagaaatgttgttaatgtattaatgatgttattaaatactgttcaagaagaacaaagtttatgcagctactgtccaaactcaaagtggcagccagttggttttgataggttgccttttggagatttctattactgcctttttttttcttactgttttattacaaacttacaaaaatatgtataaccctgttttatacaaactagtttcgtaataaaactttttcctttttttaaaatgaaaataaaaaaaa

SEQ ID NO: 5 - protein, 60 aa, Artificial Sequence
  derived from NGN2 protein
KKTRRLKANNRERNRMHNLNAALDALREVLPTFPEDAKLTKIETLRFAHNYIWALTETLR

SEQ ID NO: 6 - protein, 41 aa, Artificial Sequence
  derived from ASCL1 protein
NLGFATLREHVPNGAANKKMSKVETLRSAVEYIRALQQLLD

SEQ ID NO: 7 - DNA, 180 bp, Artificial Sequence
  derived from NGN2
aagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgc

SEQ ID NO: 8 - DNA, 123 bp, Artificial Sequence
  derived from ASCL1
aacctgggctttgccacccttcgggagcacgtccccaacggcgcggccaacaagaagatgagtaaggtggagacactgcgctcggcggtcgagtacatccgcgcgctgcagcagctgctggac

SEQ ID NO: 9 - protein, 334 aa, Homo sapiens
MTKSYSESGLMGEPQPQGPPSWTDECLSSQDEEHEADKKEDDLEAMNAEEDSLRNGGEEEDEDEDLEEEEEEEEEDDDQKPKRRGPKKKKMTKARLERFKLRRMKANARERNRMHGLNAALDNLRKVVPCYSKTQKLSKIETLRLAKNYIWALSEILRSGKSPDLVSFVQTLCKGLSQPTTNLVAGCLQLNPRTFLPEQNQDMPPHLPTASASFPVHPYSYQSPGLPSPPYGTMDSSHVFHVKPPPHAYSAALEPFFESPLTDCTSPSFDGPLSPPLSINGNFSFKHEPSAEFEKNYAFTMHYPAATLAGAQSHGSIFSGTAAPRCEIPIDNIM

SEQ ID NO: 10 - DNA, 1071 bp, Homo sapiens
atgaccaaatcgtacagcgagagtgggctgatgggcgagcctcagccccaaggtcctccaagctggacagacgagtgtctcagttctcaggacgaggagcacgaggcagacaagaaggaggacgacctcgaagccatgaacgcagaggaggactcactgaggaacgggggagaggaggaggacgaagatgaggacctggaagaggaggaagaagaggaagaggaggatgacgatcaaaagcccaagagacgcggccccaaaaagaagaagatgactaaggctcgcctggagcgttttaaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctcaggcaaaagcccagacctggtctccttcgttcagacgctttgcaagggcttatcccaacccaccaccaacctggttgcgggctgcctgcaactcaatcctcggacttttctgcctgagcagaaccaggacatgcccccccacctgccgacggccagcgcttccttccctgtacacccctactcctaccagtcgcctgggctgcccagtccgccttacggtaccatggacagctcccatgtcttccacgttaagcctccgccgcacgcctacagcgcagcgctggagcccttctttgaaagccctctgactgattgcaccagcccttcctttgatggacccctcagcccgccgctcagcatcaatggcaacttctctttcaaacacgaaccgtccgccgagtttgagaaaaattatgcctttaccatgcactatcctgcagcgacactggcaggggcccaaagccacggatcaatcttctcaggcaccgctgcccctcgctgcgagatccccatagacaatattatgtccttcgatagccattcacatcatgagcgagtcatgagtgcccagctcaatgccatatttcatgattag

SEQ ID NO: 11 - protein, 60 aa, Artificial Sequence
  derived from NEUROD1 protein
KLRRMKANARERNRMHGLNAALDNLRKVVPCYSKTQKLSKIETLRLAKNYIWALSEILRS

SEQ ID NO: 12 - DNA, 180 bp, Artificial Sequence
  derived from NEUROD1
aaattgagacgcatgaaggctaacgcccgggagcggaaccgcatgcacggactgaacgcggcgctagacaacctgcgcaaggtggtgccttgctattctaagacgcagaagctgtccaaaatcgagactctgcgcttggccaagaactacatctgggctctgtcggagatcctgcgctca